(19) United States
(12) Patent Application Publication
(10) Pub. No.: US 2017/0101435 A1
(43) Pub. Date: Apr. 13, 2017

(54) HARVESTING AND PERFUSION APPARATUS

(71) Applicant: Sarfaraz K. Niazi, Deerfield, IL (US)

(72) Inventor: Sarfaraz K. Niazi, Deerfield, IL (US)

(73) Assignee: THERAPEUTIC PROTEINS INTERNATIONAL, LLC, Chicago, IL (US)

(21) Appl. No.: 14/881,795

(22) Filed: Oct. 13, 2015

(51) Int. Cl. C07K 1/22; B01J 19/24

(52) U.S. Cl. CPC C07K 1/22; B01J 19/24; B01J 2219/24

(57) ABSTRACT

The present invention relates to an apparatus capable of harvesting a recombinant protein from a bioreactor having a porous container comprised of a chromatography medium capable of binding the recombinant protein and a method of use thereof.

### FIELD OF THE INVENTION

The present invention relates to a novel system and method of its use for harvesting and purifying proteins.

### BACKGROUND

Recombinant manufacturing of proteins involves several distinct methods:
A batch process where the protein is harvested at the end of the fermentation cycle.
A batch process where the protein is harvested repeatedly during the fermentation cycle.
A perfusion process where the culture media is replaced continuously or intermittently while cells and proteins are retained in the bioreactor.
A perfusion process where the protein and the culture media are removed from the bioreactor continuously or intermittently.
A perfusion process where the protein is removed while the culture media and cells are retained in the bioreactor.

The methodologies described above may involve a filtration step that separates either the cells or cells and proteins and returning them into bioreactor. Filtration produces a risk of contamination, breakdown of proteins and often loss of cells and/or their viability. The harvesting steps are generally followed by a centrifugation process to remove cells, which is an extremely expensive process and prone to contamination as well as loss of protein or damage to protein structure. Finally, the manufacturing of recombinant proteins requires volume reduction of the culture media prior to subjecting the protein to purification. Again, this reduction is an expensive step that may also damage protein and reduce yields.

There is a need to reduce the cost and the loss of proteins in the customary processes used in the manufacture of recombinant proteins. There remains a large unmet need to develop a device to capture the target protein non-selectively or selectively and remove it from the nutrient media or a refolding solution prior to subjecting it to customary purification processes. The present invention solves these problems by modifying the existing methods by performing protein harvesting or protein capturing prior to purification chromatography to increase the throughput of manufacturing processing without adding expensive and technically challenging modifications.

### SUMMARY OF THE INVENTION

The present invention relates to an apparatus for continuous or intermittent harvesting or perfusion useful of recombinant protein and various methods of use thereof. The apparatus comprises a first container that comprises a second porous container that retains a sufficient quantity of a chromatography resin for harvesting a desired protein from the bioreactor or a refolding chamber. The first container is capable of being raised and lowered to allow the liquid in the bioreactor or refolding chamber to flow back and forth between them. When using the apparatus to harvest protein from a bioreactor ready for harvesting or perfusion, the apparatus is connected to the bioreactor and the culture media flows into the apparatus under gravity force, being located below the level of the bioreactor. Once the apparatus fills with the nutrient media from the bioreactor, the proteins in the media bind to the chromatography resin in the second container. Then the apparatus moves upward draining the media back into the bioreactor. The apparatus continuously moves up and down repeating the cycle when the bioreactor has completed its production cycle. The apparatus can be recharged during a cycle by eluting the bound protein from the chromatography resin in the perfusion system or it can be recharged after the cycle is complete.

### BRIEF DESCRIPTION OF THE DRAWING

FIG. 1 is a side view of the apparatus attached to a bioreactor in a lowered position.

FIG. 2 is a side view of the apparatus attached to a bioreactor in an elevated position.

### DETAILED DESCRIPTION OF THE INVENTION

Traditional recombinant protein manufacturing involves growing genetically modified organisms or cells in a culture media, harvesting the target protein from the rest of the contents of the nutrient media including recombinant cells or organisms and then purifying the target protein using column chromatography.

The perfusion method of manufacturing involves replacing the nutrient media along with protein to keep cells expressing at a higher rate or removing proteins alone and replenishing the media with nutrients or any combination thereof while maintaining the cells inside the bioreactor. Customary systems involve some type of filtration of nutrient media to separate the cells from the protein and returning the cells to the bioreactor followed by adding new media. Alternatively, one can remove both cells and proteins and add new media or remove just the protein alone and replenish nutrients to the nutrient media. There are many disadvantageous to the current methods including, among other things, contamination from filtration, blockage of filters, stress to proteins resulting in loss of yield, changes in protein structure due to stress and decrease in cell viability and numbers.

The present invention comprises an apparatus used to capture a recombinant protein continuously or intermittently by using a chromatography resin that is capable of binding the target protein secreted in the nutrient media. This apparatus, when attached to a bioreactor, allows the nutrient media to drain under gravity flow into the apparatus and then returns the media to the bioreactor once the apparatus reaches a certain volume or weight. This geometric mixing allows continuous exchange of nutrient media, and the protein can be captured while the nutrient media is in the apparatus. The apparatus also comprises a sparging system to mix the media while in the apparatus increasing the efficiency of binding the target protein to the chromatography resin. All ports leading into or out of the apparatus may be protected by sterilizing filters to reduce the chance of contamination. The apparatus further comprises ports to add or remove liquid from the device. Various valves allow shutting down transfer of nutrient media from the bioreactor and allow for a process of eluting the bound protein from the chromatography resin. An eluting liquid may be introduced into the apparatus, allowing it to mix and elute and then drain the eluting liquid with the released protein from the apparatus through a port. Because the transfer of liquid is achieved by gravity flow, there is no strain on protein or cells that may come from the use of peristaltic pumps in the transfer of nutrient media to and from the apparatus.

The apparatus can be permanently attached to one bioreactor or multiple bioreactors and these bioreactors may be single-use or multiple use systems. The separation of proteins can be achieved continuously or intermittently.

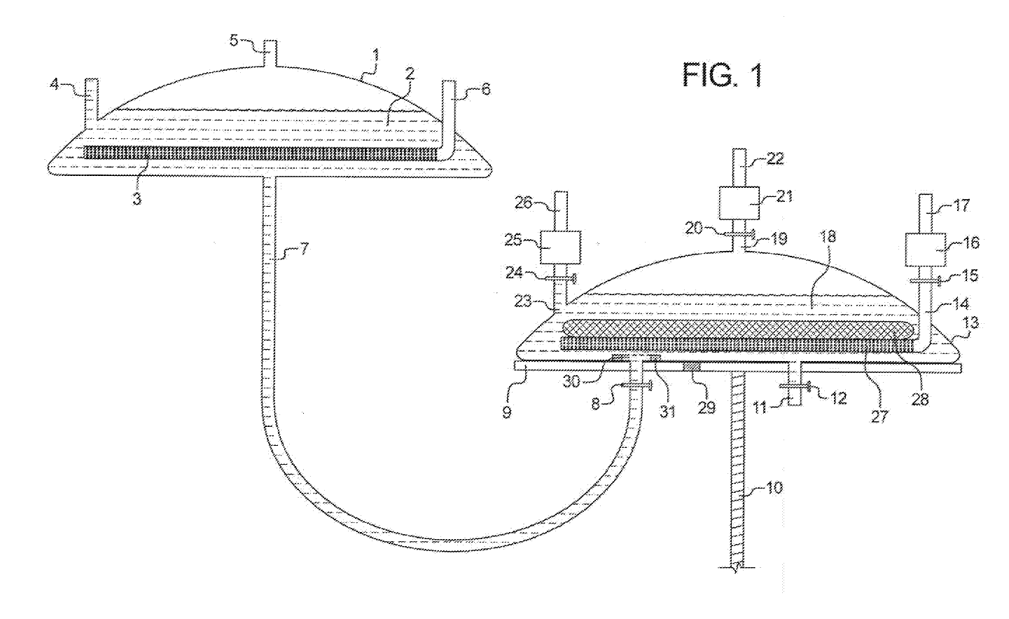

FIG. 1 describes a side view of a bioreactor connected to the apparatus that has been lowered to receive nutrient media. In one embodiment, the bioreactor (1) is filled with a nutrient medium (2) and the cultured organism, engineered to make the desired protein, is aerated by a gas sparger (3) and the air or gas is introduced into the sparger through the gas inlet port (6). Media can be added through the media inlet port (4) and any gas that builds up in the bioreactor can be released through the gas outlet port (5). When the concentration of cultured organism reaches a desired level, the nutrient media is transferred from the bioreactor to the apparatus (13) via the connector tube (7) by opening the valve (8).

In order to move the nutrient media from the bioreactor to the apparatus of the present invention, the apparatus is placed on a movable platform (9) and it is raised and lowered by a platform moving device (10). The platform is lowered to allow the media to drain into the apparatus, the protein binds to the resin in the permeable pouch (28) and then the platform is raised to drain the media back into the bioreactor to continue the production process. The liquid outlet port (11) can be opened if the contents in the apparatus are to be removed rather than returned to the bioreactor by opening the liquid outlet valve (12). Gas can be introduced into the apparatus via the gas inlet port (17) through an optional filter (16) through the tube (14) to a gas sparger (27) to aid in circulating the media (18) through the permeable pouch (28) containing the chromatography resin, increasing the efficiency of protein capture. This tube can be sealed during certain aspects of the method by closing valve (15). Gas can be released from the apparatus via the gas outlet tube (19) through an optional filter (21) and out through the port {22). The tube can be opened and closed via valve (20). Liquid can be introduced directly into the apparatus, e.g., eluting liquid, through liquid inlet port (26) through a sterilizing filter (5) through tube (23) into the apparatus. This inlet can be opened and closed via a valve (24). The amount of liquid that is allowed to flow into the apparatus can be controlled using a weight sensor (29). Optionally, for certain embodiments, a membrane (30) is placed over the opening to the apparatus from the bioreactor and one can vibrate the liquid exiting or entering the apparatus through the connecting tube (7).

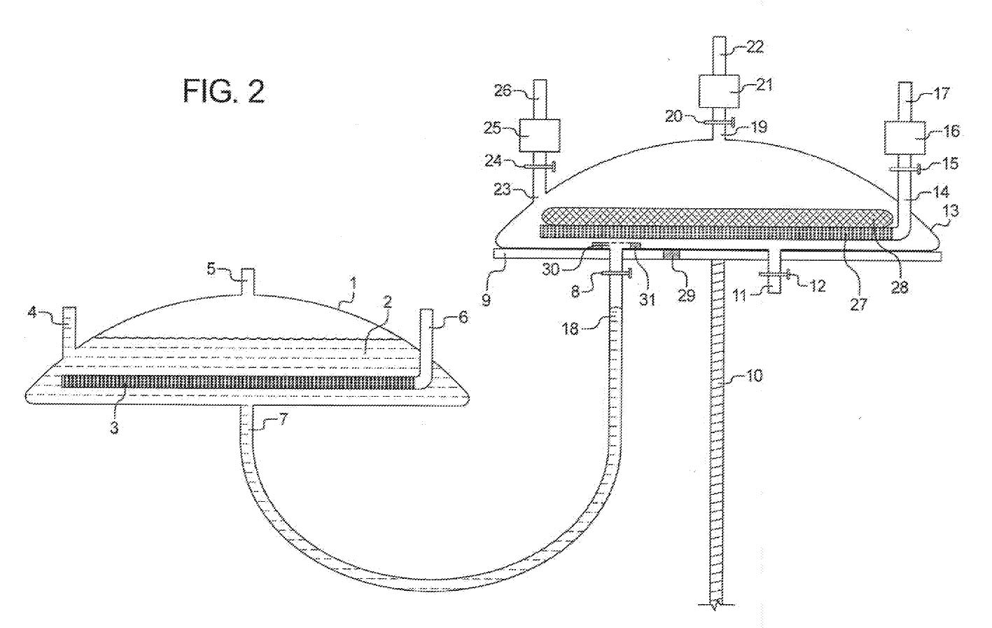

FIG. 2 illustrates a side view of a bioreactor connected to the apparatus raised above the level of the bioreactor and from which the nutrient media has been drained back to the bioreactor.

The apparatus depicted in the figures is generally used during the bioreactor cycle. The quantity of chromatography resin in the apparatus will depend on the capacity of binding required. The resin is contained inside a perforated pouch to contain the resin inside the apparatus without any loss of resin as the liquid is removed from the device.

Examples of resin that may be used in the present invention include, but are not limited to: Dual Affinity Polypeptide technology platform; Protein A; Protein G; stimuli responsive polymers enable complexation and manipulation of proteins; mixed mode sorbents; ion exchange media; hydrophobic charge induction chromatography, such as MEP, and Q and S HyperCel; Monoliths, such as Convective Interaction Media monolithic columns; simulated moving beds, such as BioSMB; single domain camel-derived (camelid) antibodies to IgG, such as CaptureSelect; inorganic ligands, including synthetic dyes, such as Mabsorbent A1P and A2P; Expanded bed adsorption chromatography systems, such as the Rhobust platform; ultra-durable zirconia oxide-bound affinity ligand chromatography media; Fc-receptor mimetic ligand; ADSEPT (ADvanced SEParation Technology); membrane affinity purification system; custom-designed peptidic ligands for affinity chromatography; protein A- and G-coated magnetic beads; affinity purification methods based on expression of proteins or MAbs as fusion proteins with removable portion (tag) having affinity for chromatography media, such as histidine tags; protein A alternatives in development; plug-and-play solutions with disposable components; affinity chromatography media; lectin chromatography media; and immunoaffinity chromatography media.

The apparatus will typically be a single-use device that comes installed with sparger, a porous pouch containing binding resin, filters and valves, ready to be attached to a bioreactor. The porous pouch can be comprised of a nylon mesh with pores smaller than the size of resin particles, generally 50-200 microns. Therefore, the pouch will have a permeability to retain any particle larger than 50 microns inside the pouch.

In a typical operation for perfusion, the apparatus will be attached to the bioreactor using flexible tubing that is connected in an sterile manner. At first, the valve for entry of nutrient media to the apparatus will be closed. The apparatus residing on a movable platform is lowered to a level below the level of the bioreactor, the valve for the entry of nutrient media to the device is opened while at the same time, gas is allowed to enter the device causing mixing of the nutrient media as it enters the apparatus. Once the quantity of nutrient media reaches a certain volume or weight as measured by the weight sensor, the platform holding the apparatus rises to a level above the bioreactor causing the nutrient media to drain back into the bioreactor. Once the media is drained, either completely or partially, the platform is lowered in response to a signal from the weight sensor. The cycle is continuously or intermittently repeated until the resin becomes fully saturated with protein. At this point, the valve of nutrient media entry into the device is closed after the nutrient media has been drained back into the bioreactor and an eluting liquid is added to the device through the liquid inlet. The eluting liquid is allowed to remain inside the device for a time sufficient to allow removal of protein from the resin. The eluting liquid is then drained through liquid outlet and then the nutrient media valve is opened again and the cycle repeated as often as necessary.

Use of the apparatus of the present invention solves several problems associated with isolating proteins from a bioreactor, e.g., cost, time and degradation of protein. The present invention simplifies the removal of proteins from a bioreactor on a continuous or intermittent basis by combining several steps. In particular, the present apparatus may be used for harvesting a recombinant protein directly from the nutrient media without first removing the cells. The nutrient media containing host cells and target protein are subjected to a non-specific or specific treatment with chromatography resin or a combination of chromatography resin contained in the apparatus that binds all or substantially all of the target protein. This step is followed by removing the target protein from the chromatography resin by simply eluting the protein directly in the apparatus and then removing the protein from the apparatus through a liquid port. The present invention thus obviates a major hurdle in the harvesting of proteins that involves filtering out host cells using a fine filter, not larger than 5 micron, to retain host cells such as Chines Hamster Ovary Cells, saving time and money. When a large volume of media is used, this process can take a very long time, adds substantial cost of due to the use of filters, pumps, containers and space management. This step is then generally followed by a concentrating step wherein the volume of nutrient media is reduced most to one-tenth its volume using a cross-flow or micro filtration process, which takes a very long time to complete and again adds substantial cost of equipment, manpower and in some instances causes degradation of target protein. The present invention combines these two steps into one simple step.

In the present invention, those peculiar characteristics of target proteins are exploited to separate them from the rest of the mixture by a non-specific binding to a chromatography resin or a mixture of chromatography resins. Obviously, such non-specific capture of target proteins would also capture other components of the mixture and that only requires using a much larger quantity of chromatography resin or a specific type of chromatography resin that might have specific affinity for the target protein. The removal of protein-chromatography resin complex is a much simpler process than the removal of host cells or reduction in the volume of mixture. It is noteworthy that the slowest of all processes would be filtration but even the much larger pore size filter can be used and since the purpose is to collect the filtrate, not the eluate, the cost of manufacturing is lowered substantially.

In a second embodiment of the present invention, one can concentrate proteins in stages of the purification of the protein other than from the bioreactor. Some proteins require refolding after their initial purification and this refolding takes place in a very dilute solution. These solutions are of high purity and can be readily filtered, but it is most frequently seen that the filtration of a refolded solution results in a substantial loss of protein due to degradation. The present invention resolves this problem by removing all or substantially all of protein solution from the refolding solution, removing the buffers and reconstituting the protein eluted from the chromatography resin-protein complex for further purification.

In a third embodiment, the present invention can be applied to separation of any protein solution including industrial production of proteins.

In a fourth embodiment, the present invention eliminates the need for costly filtration processes for manufacturing of proteins because a concentration step is not needed.

In a fifth embodiment, the present invention provides a means of continuously removing expressed protein from a nutrient media to enhance the level of expression that may be depressed because of the higher concentration of protein in the mixture.

In a sixth embodiment, the present invention provides a means of continuously removing expressed protein from a nutrient media to reduce the toxicity of the expressed protein to host cells and thus prolonging the cycles of expression, thus substantially increasing the yields of production. In a biological system, a particular protein is expressed only in a specific subcellular location, tissue or cell type, during a defined time period, and at a particular quantity level. This is known as a spatial, temporal, and quantitative expression. Recombinant protein expression often introduces a foreign protein into a host cell, expressing the protein at levels significantly higher than the physiological level of the protein in its native host and at a time the protein is not needed. The over-expressed recombinant protein will perform certain functions in the host cell if the protein is expressed soluble and functional. The function of the expressed recombinant protein is often not needed by the host cell. In fact, the function of the protein may be detrimental to the proliferation and differentiation of the host cell. The observed phenotypes of the host cells under these conditions are slow growth rate and low cell density. In some cases, the recombinant protein causes death of the host cell. These phenomena are described as protein toxicity and the recombinant proteins are called toxic proteins. Therefore, it is prudent to transport the protein out of the cell as soon as possible such as in the case of the present invention by binding to a chromatography resin.

In a seventh embodiment, the present invention provides a means of increasing the chemical stability of certain expressed proteins by binding them to a chromatography resin as soon as they are expressed, as the chemicals are always less stable in a solution form than in a solid form or in this case a complex form. By stabilizing the protein, one can substantially improve the yield of production. The very nature of the recombinant product makes it unstable. Instability of a recombinant protein can be separated into either physical instability issues or chemical instability issues. Physical instability can be related to such things as denaturation of the secondary and tertiary structure of the protein; adsorption of the protein onto interfaces or excipients; and aggregation and precipitation of the protein. In most biopharmaceutical processes, additives are used to improve the physical stability of a protein. The addition of salts can significantly decrease denaturation and aggregation by the selective binding of ions to the protein. Polyalcohols can also be used to stabilize the protein by selective solvation. Finally, surfactants are often used to prevent the adsorption of proteins at the surface, although there is a fine line between the amount of surfactant needed to prevent adsorption and the amount needed to denature a product. In addition, excipients are often used to prevent aggregation. Chemical instability of a protein product results in the formation of a new chemical entity by cleavage or by new bond formation. Examples of this type of instability would be deamidation, proteolysis and racemization. There are some more obvious choices to improve the chemical instability, such as modulation of pH, the use of low temperatures for storage and processing, and the addition of preservatives. In the process of recombinant manufacturing where proteins are secreted into media, there are two methods widely used. In one method of batch processing, the proteins are harvested at the end of the cycle that might be as long as several weeks of continuous expression; while many proteins would survive the 37 C environment for that length of time, many would degrade over period of time. By capturing the proteins through formation of chromatography resin-protein complex, the stability of and thus the yield of production can be increased since in the complex stage, the molecules are immobilized and thus less likely to decompose. While many proteins may decompose by adsorbing to various surfaces, the interaction between a chromatography resin and protein is of a different nature as evidenced by the use of chromatography resins in the purification of proteins whereby high degree of stability is maintained when eluting from a chromatography resin column. In another situation, where a apparatus is used for the upstream production of recombinant proteins, a portion of nutrient media is replaced with fresh media and the media removed is filtered of host cells, reduced in volume and either stored at a lower temperature or processed with downstream processing. This technique also adds substantial cost to production in media and its handling. By passing the media through a column containing the chromatography resin, which can be replaced with fresh chromatography resin periodically, the expressed protein can be removed readily without affecting the total count of viable host cells. While the chromatography resin might also absorb some of the nutrients, these can be easily replaced in a fed-batch culture system.

In the eighth embodiment, the present invention combines several steps of upstream and downstream; the chromatography resin-protein complex as contained in the container of the device is ready for downstream processing that can be accomplished by loading the device onto a column. This can save substantial time for loading. This prolonged delay can also be detrimental to the stability of target protein.

In the ninth embodiment, the present invention offers to eliminate a very laborious and expensive step of first stage filtration or other means of separating the protein harvested. By using a device to contain the chromatography resin, all steps generally required to remove chromatography resin, such as decanting, centrifugation (low speed), and filtration (coarse) can be avoided altogether. The containers can be strung together so that these are simply removed by picking up the end of the string at one end. The porous resin container can be removed from the apparatus and packed directly into a column for elution as if this were loose chromatography resin.

In the tenth embodiment, the present invention allows one to adjust the physicochemical characteristics of the nutrient media to achieve optimal binding of proteins with chromatography resin improving the yield.

In the eleventh embodiment, the present invention allows for the use of a mixed-bed chromatography resin that may contain an ionic chromatography resin, a hydrophobic chromatography resin and an affinity chromatography resin all used together to optimize the efficiency of harvesting. It is well established that the use of ionic chromatography resins does not allow complete capture of proteins because of the logarithmic nature of ionization. However, a combination of chromatography resins used in the present invention allows for a more complete recovery of target proteins. Since the purpose of reaction at the chromatography resin-protein complexation stage is to harvest and not purify the protein, the calculations like chromatography plates for purification are not important and neither is the particle size of the chromatography resin allowing use of the cheapest chromatography resin available. Any lack of efficiency in capturing proteins can be readily adjusted by increasing the quantity of chromatography resin. The chromatography resin can be used repeatedly after washing of the proteins and sanitizing the chromatography resin.

In the twelfth embodiment, the present invention describes a novel method of protein purification wherein the loading of purification column is avoided. The protein-chromatography complex in the device is already loaded. Often it takes hours and days to load a column, these steps are obviated in the use of the claimed apparatus.

In the thirteenth embodiment, the present invention describes a method of keeping the chromatography resin binding the protein separate from the nutrient media inside a bioreactor and thus allowing separation of wasted nutrient media and cells by simply draining the bioreactor. This eliminates at least three steps in downstream processing, viz., filtration of culture broth to remove cells, cross-flow filtration to reduce the volume of broth and finally loading of protein solution onto a separation column.

In the fourteenth embodiment, the present invention describes an apparatus capable of containing a chromatography resin capable of binding the proteins and it is added in a device that is capable of floating in the nutrient media. Furthermore, the buoyance of the device can be adjusted by applying various weight to it to assure that the device is submerged in the nutrient media at different levels to maximize the binding of the protein to the chromatography resin. The buoyancy device may be comprised of a continuous thin walled plastic body enclosing a substantially hollow interior, or of polymeric foam and encircling the container. The buoyancy device may be comprised of cork and encircling the container. Alternatively, the buoyancy device may be inflatable and inflated to different pressures to produce buoyancy.

The embodiments described above do not in any way comprise all embodiments that are possible using the present invention and one with ordinary skills in the art would find many more applications specific to a complex process or even in those processes where such needs might not be immediately apparent.

The present invention is significantly different from a typical separative type bioreactor. In the present invention, the bioreactor is operated separate from the apparatus, alleviating the need to sterilize the resin. It also allows for the removal of the protein from the resin during the production cycle in the bioreactor. It also allows the conditions in the apparatus to be adjusted separate from the bioreactor, such as temperature or binding conditions, before returning the media to the bioreactor. Moreover, different resins can be used during the course of purification.

A physical model of the transport of liquids from one container to another is analogous to a physical clearance and equilibration model. For the contents to be declared as homogenously mixed, the contents should be moved back and forth sufficient times to achieve homogeneity. The rate of equilibration to achieve homogeneity is easily calculated by the rate constant of the liquid transfer to and from each container. Assuming that the liquid in two bioreactors is transferred back and forth at the same rate simultaneously, then the rate constant for equilibration is simply the ratio of the volume transferred per unit of time. As an example, if 10 L of liquid is transferred between two bioreactors per minute, each containing 100 L of liquid, K value is 0.1 and based on the exponential nature of equilibration, the half-life of equilibration would be 0.693/K or 6.93 minutes. To achieve a 99% equilibration, approximately seven half lives are needed or about 50 minutes of continuous mixing comprising transporting within each minute 10 L of liquid from one container to the other container.

Examples of cells that can be used in the operation of the bioreactor, include, but are not limited to: Chinese hamster ovary (CHO), mouse myeloma cells, M0035 (NSO cell line), hybridomas (e.g., B-lymphocyte cells fused with myeloma tumor cells), baby hamster kidney (BHK), monkey COS, African green monkey kidney epithelial (VERO), mouse embryo fibroblasts (NIH-3T3), mouse connective tissue fibroblasts (L929), bovine aorta endothelial (BAE-1), mouse myeloma lymphoblastoid-like (NSO), mouse B-cell lymphoma lymphoblastoid (WEHI 231), mouse lymphoma lymphoblastoid (YAC 1), mouse fibroblast (LS), hepatic mouse (e.g., MC/9, NCTC clone 1469), and hepatic rat cells (e.g., ARL-6, BRL3A, H4S, Phi 1 (from Fu5 cells)). Human cells include retinal cells (PER-C6), embryonic kidney cells (HEK-293), lung fibroblasts (MRC-5), cervix epithelial cells (HELA), diploid fibroblasts (WI38), kidney epithelial cells (HEK 293), liver epithelial cells (HEPG2), lymphoma lymphoblastoid cells (Namalwa), leukemia lymphoblastoid-like cells (HL60), myeloma lymphoblastoid cells (U 266B1), neuroblastoma neuroblasts (SH-SY5Y), diploid cell strain cells (e.g., propagation of poliomyelitis virus), pancreatic islet cells, embryonic stem cells (hES), human mesenchymal stem cells (MSCs, which can be differentiated to osteogenic, chondrogenic, tenogenic, myogenic, adipogenic, and marrow stromal lineages, for example), human neural stem cells (NSC), human histiocytic lymphoma lymphoblastoid cells (U937), and human hepatic cells such as WRL68 (from embryo cells), PLC/PRF/5 (i.e., containing hepatitis B sequences), Hep3B (i.e., producing plasma proteins: fibrinogen, alpha-fetoprotein, transferrin, albumin, complement C3 and/or alpha-2-macroglobulin), and HepG2 (i.e., producing plasma proteins: prothrombin, antithrombin III, alpha-fetoprotein, complement C3, and/or fibrinogen).

Cells from insects (e.g., baculovirus and Spodoptera frugiperda ovary (Sf21 cells produce SD line)) and cells from plants or food, may also be cultured in accordance with the invention. Cells from sources such as rice (e.g., Oryza sativa, Oryza sativa cv Bengal callus culture, and Oryza sativa cv Taipei 309), soybean (e.g., Glycine max cv Williams 82), tomato (Lycopersicum esculentum cv Seokwang), and tobacco leaves (e.g., Agrobacterium tumefaciens including Bright Yellow 2 (BY-2), Nicotiana tabacum cv NT-1, N. tabacum cv BY-2, and N. tabacum cv Petite Havana SR-1) are illustrative examples.

Bacteria, fungi, or yeast may also be cultured in accordance with the invention. Illustrative bacteria include Salmonella, Escherichia coli, Vibrio cholerae, Bacillus subtilis, Streptomyces, Pseudomonas fluorescens, Pseudomonas putida, Pseudomonas sp, Rhodococcus sp, Streptomyces sp, and Alcaligenes sp. Fungal cells can be cultured from species such as Aspergillus niger and Trichoderma reesei, and yeast cells can include cells from Hansenula polymorpha, Pichia pastoris, Saccharomyces cerevisiae, S. cerevisiae crossed with S. bayanus, S. cerevisiae crossed with LAC4 and LAC1-2 genes from K. lactis, S. cerevisiae crossed with Aspergillus shirousamii, Bacillus subtilis, Saccharomyces diastasicus, Schwanniomyces occidentalis, S. cerevisiae with genes from Pichia stipitis, and Schizosaccharomyces pombe.

A variety of different products may also be produced in accordance with the invention. Illustrative products include proteins (e.g., antibodies and enzymes), vaccines, viral products, hormones, immunoregulators, metabolites, fatty acids, vitamins, drugs, antibiotics, cells, and tissues. Non-limiting examples of proteins include human tissue plasminogen activators (tPA), blood coagulation factors, growth factors (e.g., cytokines, including interferons and chemokines), adhesion molecules, Bcl-2 family of proteins, polyhedrin proteins, human serum albumin, scFv antibody fragment, human erythropoietin, mouse monoclonal heavy chain 7, mouse IgG.sub.2b/k, mouse IgG1, heavy chain mAb, Bryondin 1, human interleukin-2, human interleukin-4, ricin, human .alpha.1-antitrypisin, biscFv antibody fragment, immunoglobulins, human granulocyte, stimulating factor (hGM-CSF), hepatitis B surface antigen (HBsAg), human lysozyme, IL-12, and mAb against HBsAg. Examples of plasma proteins include fibrinogen, alpha-fetoprotein, transferrin, albumin, complement C3 and alpha-2-macroglobulin, prothrombin, antithrombin III, alpha-fetoprotein, complement C3 and fibrinogen, insulin, hepatitis B surface antigen, urate oxidase, glucagon, granulocyte-macrophage colony stimulating factor, hirudin/desirudin, angiostatin, elastase inhibitor, endostatin, epidermal growth factor analog, insulin-like growth factor-1, kallikrein inhibitor, .alpha.1-antitrypsin, tumor necrosis factor, collagen protein domains (but not whole collagen glycoproteins), proteins without metabolic byproducts, human albumin, bovine albumin, thrombomodulin, transferrin, factor VIII for hemophilia A (i.e., from CHO or BHK cells), factor VIIa (i.e., from BHK), factor IX for hemophilia B (i.e., from CHO), human-secreted alkaline phosphatase, aprotinin, histamine, leukotrienes, IgE receptors, N-acetylglucosaminyltransferase-III, and antihemophilic factor VIII.

Enzymes may be produced from a variety of sources using the invention. Non-limiting examples of such enzymes include YepACT-AMY-ACT-X24 hybrid enzyme from yeast, Aspergillus oryzae .alpha.-amylase, xylanases, urokinase, tissue plasminogen activator (rt-PA), bovine chymosin, glucocerebrosidase (therapeutic enzyme for Gaucher's disease, from CHO), lactase, trypsin, aprotinin, human lactoferrin, lysozyme, and oleosines.

Vaccines also may be produced using the invention. Non-limiting examples include vaccines for prostate cancer, human papilloma virus, viral influenza, trivalent hemagglutinin influenza, AIDS, HIV, malaria, anthrax, bacterial meningitis, chicken pox, cholera, diphtheria, haemophilus influenza type B, hepatitis A, hepatitis B, pertussis, plague, pneumococcal pneumonia, polio, rabies, human-rabies, tetanus, typhoid fever, yellow fever, veterinary-FMD, New Castle's Disease, foot and mouth disease, DNA, Venezuelan equine encephalitis virus, cancer (colon cancer) vaccines (i.e., prophylactic or therapeutic), MMR (measles, mumps, rubella), yellow fever, Haemophilus influenzae (Hib), DTP (diphtheria and tetanus vaccines, with pertussis subunit), vaccines linked to polysaccharides (e.g., Hib, Neisseria meningococcus), Staphylococcus pneumoniae, nicotine, multiple sclerosis, bovine spongiform encephalopathy (mad cow disease), IgG1 (phosphonate ester), IgM (neuropeptide hapten), SIgA/G (Streptococcus mutans adhesin), scFv-bryodin 1 immunotoxin (CD-40), IgG (HSV), LSC (HSV), Norwalk virus, human cytomegalovirus, rotavirus, respiratory syncytial virus F, insulin-dependent autoimmune mellitus diabetes, diarrhea, rhinovirus, herpes simplex virus, and personalized cancer vaccines, e.g., for lymphoma treatment (i.e., in injectable, oral, or edible forms). Recombinant subunit vaccines also may be produced, such as hepatitis B virus envelope protein, rabies virus glycoprotein, E. coli heat labile enterotoxin, Norwalk virus capsid protein, diabetes autoantigen, cholera toxin B subunit, cholera toxin B an dA2 subunits, rotavirus enterotoxin and enterotoxigenic E. coli, fimbrial antigen fusion, and porcine transmissible gastroenteritis virus glycoprotein S.

Viral products also may be produced. Non-limiting examples of viral products include sindbis, VSV, oncoma, hepatitis A, channel cat fish virus, RSV, corona virus, FMDV, rabies, polio, reo virus, measles, and mumps.

Hormones also may be produced using the invention. Non-limiting examples of hormones include growth hormone (e.g., human growth hormone (hGH) and bovine growth hormone), growth factors, beta and gamma interferon, vascular endothelial growth factor (VEGF), somatostatin, platelet-derived growth factor (PDGF), follicle stimulating hormone (FSH), luteinizing hormone, human chorionic hormone, and erythropoietin.

Immunoregulators also may be produced. Non-limiting examples of immunoregulators include interferons (e.g., beta-interferon (for multiple sclerosis), alpha-interferon, and gamma-interferon) and interleukins (such as IL-2).

Metabolites (e.g., shikonin and paclitaxel) and fatty acids (i.e., including straight-chain (e.g., adipic acid, Azelaic acid, 2-hydroxy acids), branched-chain (e.g., 10-methyl octadecanoic acid and retinoic acid), ring-including fatty acids (e.g., coronaric acid and lipoic acid), and complex fatty acids (e.g., fatty acyl-CoA)) also may be produced.

The containers useful in the various embodiments of the invention may be of any size suitable for containing a liquid. For example, the container may have a volume between 1-40 L, 40-100 L, 100-200 L, 200-300 L, 300-500 L, 500-750 L, 750-1,000 L, 1,000-2,000 L, 2,000-5,000 L, or 5,000-10,000 L. In some instances, the container has a volume greater than 1 L, or in other instances, greater than 10 L, 20 L, 40 L, 100 L, 200 L, 500 L, or 1,000 L. Volumes greater than 10,000 L are also possible. Preferably, the container volume will range between about 1 L and 1000 L, and more preferably between about 5 L and 500 L, and even more preferably between 5 L and 200 L.

The components of the bioreactors and other devices described herein, which come into contact with the culture medium or products provided thereby, desirably comprise biocompatible materials, more desirably biocompatible polymers, and are preferably the materials can be sterilized.

It should also be understood that many of the components described herein also are desirably flexible, e.g., the containers desirably comprise flexible biocompatible polymer containers (such as collapsible bags), with the conduits also desirably comprising such biocompatible polymers. The flexible material is further desirably one that is USP Class VI certified, e.g., silicone, polycarbonate, polyethylene, and polypropylene. Non-limiting examples of flexible materials include polymers such as polyethylene (e.g., linear low density polyethylene and ultra low density polyethylene), polypropylene, polyvinylchloride, polyvinyldichloride, polyvinylidene chloride, ethylene vinyl acetate, polycarbonate, polymethacrylate, polyvinyl alcohol, nylon, silicone rubber, other synthetic rubbers and/or plastics. If desired, portions of the flexible container may comprise a substantially rigid material such as a rigid polymer (e.g., high density polyethylene), metal, and/or glass.

Desirably the containers comprise biocompatible materials, more desirably biocompatible polymers. When collapsible containers are selected for use, the container may be supported by or may line an inner surface of a support structure, e.g., the outer support housing having container-retaining sidewalls. However, the invention may be practiced using non-collapsible or rigid containers or conduits.

The containers may have any thickness suitable for retaining the culture medium within, and may be designed to have a certain resistance to puncturing during operation or while being handled. For example, the walls of a container may have a total thickness of less than or equal to 250 mils (1 mil is 25.4 micrometers), less than or equal to 200 mils, less than or equal to 100 mils, less than or equal to 70 mils (1 mil is 25.4 micrometers), less than or equal to 50 mils, less than or equal to 25 mils, less than or equal to 15 mils, or less than or equal to 10 mils. In certain embodiments, the container may include more than one layer of material that may be laminated together or otherwise attached to one another to impart certain properties to the container. For instance, one layer may be formed of a material that is substantially oxygen impermeable. Another layer may be formed of a material to impart strength to the container. Yet another layer may be included to impart chemical resistance to fluid that may be contained in the container.

It thus should be understood that a container may be formed of any suitable combinations of layers. The container (e.g., collapsible bag) may include, for example, 1 layer, greater than or equal to 2 layers, greater than or equal to 3 layers, or greater than equal to 5 layers of material(s). Each layer may have a thickness of, for example, less than or equal to 200 mils, less than or equal to 100 mils, less than or equal to 50 mils, less than or equal to 25 mils, less than or equal to 15 mils, less than or equal to 10 mils, less than or equal to 5 mils, or less than or equal to 3 mils, or combinations thereof.

In addition, the container preferably is seamless in order to improve its strength and avoid deposition of growing cells in the media.

All methods used for raising or lowering the bioreactors require a mechanical method and several methods are readily available in the art. These may include using an electrical motor, a hydraulic device, an air-driven device or any other such method, the choice of which is not limiting in the present invention.

All references, including publications, patent applications, and patents, cited herein are hereby incorporated by reference to the same extent as if each reference were individually and specifically indicated to be incorporated by reference and were set forth in its entirety herein.

In addition, the apparatus may further comprise a computerized or programmable apparatus for controlling the valves, opening and closing automatically; or for raising and lowering the platform according to the weight readings of the weight sensor.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

What is claimed is:

1. An apparatus to harvest expressed protein from culture media comprising:
a. A first container with inner volume, a top surface, and a bottom surface;
b. A first liquid port in the bottom surface and comprising a valve or clamp to open or close the first liquid port for inlet and outlet of liquid;
c. A second liquid port at the bottom surface with a valve or clamp to open or close the second liquid port in the first container for the outlet of liquid;
d. A third liquid port at the top surface comprising a valve or a clamp to close or open the third liquid port for inlet of liquid and an optional filter;
e. A first gas port at the top surface comprising
i. A valve or a clamp to close or open the first gas port for inlet of gas;
ii. A sparging rod disposed at the bottom of the first container; and
iii. an optional filter;
f. A second gas port at the top surface comprising a valve or a clamp to close or open the second gas port for outlet of gas and an optional filter;
g. A second container with an inner volume comprised of a porous material containing a binding resin, wherein the second container is disposed inside the first container;
h. A flexible tube connecting the first liquid port in bottom surface of the first container to a bioreactor; and
i. A movable platform to support the first container capable of moving the first container up and down.

2. The apparatus of claim 1, further comprising a weight sensor attached to the movable platform.

3. The apparatus of claim 2, wherein the weight sensor is connected to a device capable of automatically controlling the movable platform to move up or down based on a pre-determined weight limit of the first container.

4. The apparatus of claim 1, further comprising a filter disposed inside the first container and attached to the first liquid port and a vibrator attached to the filter attached to the first liquid port.

5. The apparatus of claim 1, where the first container is a flexible bag.

6. The apparatus of claim 1, wherein the second container is a flexible pouch.

7. The apparatus of claim 1, wherein the second container is buoyant.

8. The apparatus of claim 1, wherein the second container is comprised of nylon mesh, a flexible perforated plastic, wood or metal.

9. The apparatus of claim 1, wherein the second container comprises a plurality of pores having a size ranging from 5 microns to 50 microns.

10. The apparatus of claim 1, wherein the second container comprises a plurality of pores having a size ranging from 50 to 100 microns.

11. The apparatus of claim 1, wherein the second container comprises a plurality of pores having a size ranging from 100 to 300 microns.

12. The apparatus of claim 1, wherein the binding resin is Protein A.

13. The apparatus of claim 1, wherein the binding resin is a mixed bed resin.

14. The apparatus of claim 13, wherein the resin is selected from ion exchange resins, hydrophobic chromatography resins, and affinity resins.

15. A method of harvesting an expressed protein from a culture media produced in a bioreactor comprising:
a. Providing the apparatus of claim 1;
b. Connecting the first liquid port to a bioreactor wherein the protein is produced;
c. Opening the first gas and second gas ports;
d. Starting flow of gas through first gas port to begin gas flow through the sparging tube;
e. Lowering the platform supporting the apparatus to below the level of the bioreactor;
f. Allowing gravity flow of culture media in the bioreactor into the first container;
g. Raising the platform supporting the apparatus when the weight of the apparatus reaches a predetermined level, to above the level of the bioreactor;
h. Allowing gravity flow of the culture media from the first container back into the bioreactor;
i. Repeat steps (e) to (h) for a pre-determined number of times based on binding capacity of the resin disposed of in the second porous container;
j. Raising the platform to a level above the level of the bioreactor and allowing culture medium in the first container drain into the bioreactor;
k. Closing the first liquid port between the bioreactor and the apparatus;
l. Opening the second liquid port and introducing an eluting liquid capable of eluting the protein from the resin in the second porous container;
m. Opening the third liquid port and draining the eluting liquid from the apparatus for further processing;
n. Closing the third liquid port;
o. Introducing a washing liquid through the second liquid port into the apparatus;
P. Allowing the washing liquid to sit for a period of time;
q. Opening the third liquid port and draining the washing liquid from the apparatus;
r. Repeat steps (p) to (r) for a pre-determined number of times and discarding the washing liquid;
s. Closing the third liquid port;
t. Opening the first liquid port; and
u. Repeating steps (e) to (t) for a pre-determined number of times to harvest part or all of the protein produced in the bioreactor.

16. The method of claim 15, wherein the method is used at the end of a protein expression cycle in the bioreactor.

17. The method of claim 15, wherein the method is used during the production of the protein in the bioreactor.

18. The method of claim 15, wherein the said method is used continuously or intermittently during the production of the protein in the bioreactor.

19. The method of claim 15, wherein the binding of the protein to resin stabilizes the protein, thereby improving yield.

20. The method of claim 15, wherein the binding of the protein to resin improves the yield of production due to removing a toxic protein from the media.

21. A method for refolding proteins comprising:
a. Obtaining a purified protein in need of refolding;
b. Diluting the protein in a refolding buffer;
c. Introducing the protein in said buffer into the apparatus of claim 1 through the second liquid port;
d. Allowing the protein to bind to the resin in the second porous container;
e. Opening the third liquid port and draining the refolding buffer from the apparatus;
f. Closing the third liquid port;
g. Introducing a washing liquid through the second liquid port into the apparatus;
h. Allowing the washing liquid to sit for a period of time;
i. Opening the third liquid port and draining the washing liquid from the apparatus;
j. Repeat steps (g) to (i) for a pre-determined number of times and discarding the washing liquid;
k. Opening the second liquid port and introducing an eluting liquid capable of eluting the refolded protein from the resin;
l. Opening the third liquid port and draining the eluting liquid from the apparatus for further processing;
m. Closing the third liquid port.
